(19) United States
(12) Patent Application Publication
(10) Pub. No.: US 2008/0004283 A1
(43) Pub. Date: Jan. 3, 2008

(54) Pharmaceutical Compositions for the Treatment of Cellulite

(75) Inventors: Massimiliana Landini, Pomezia (IT); Sandro Giuliani, Bagno di Ripoli (IT); Alessandro Giolitti, Firenze (IT)

Correspondence Address: YOUNG & THOMPSON, 745 SOUTH 23RD STREET, 2ND FLOOR, ARLINGTON, VA, 22202, US

(73) Assignee: MENARINI RICERCHE S.P.A., POMEZIA (IT)

(21) Appl. No.: 11/792,912

(22) PCT Filed: Dec. 6, 2005
(86) PCT No.: PCT/EP05/13041

§ 371 Date: Jun. 13, 2007

(51) Int. Cl. A61K 31/4166; A61K 31/437; A61K 31/444; A61K 31/4709; A61K 31/501; A61K 31/519; A61K 8/49; A61P 17/00; A61Q 19/06

(52) U.S. Cl. 514/252.06; 514/267; 514/300; 514/312; 514/334; 514/392

(57) ABSTRACT

There are disclosed pharmaceutical compositions for topical administration or for use in mesotherapy, containing a PDE3 inhibitor as active ingredient, and their use in the treatment of cellulite.

The present invention relates to pharmaceutical formulations for the topical or mesotherapeutic treatment of cellulite, which contain as active ingredient a PDE3 inhibitor, optionally associated with other substances having anti-cellulite activity.

### BACKGROUND TO THE INVENTION

Cellulite, or “oedematous-fibrosclerotic panniculopathy”, is a disorder that affects the hypodermis, a tissue situated below the dermis, which has a mainly adipose nature. Cellulite almost exclusively affects women, and is suffered by approximately 80-85% of the post-adolescent female population. Subcutaneous adipose tissue represents 25% of body weight in women, and performs 3 basic functions: a) it provides physical and mechanical protection; b) it releases lipid and protein substances involved in lipid metabolism; and c) it performs an endocrine and paracrine action.

Statistically, this blemish affects the majority of Caucasian women. The problem is much less frequent in women of other races. The women most susceptible to the disorder are “Mediterranean” women, generally due to the fact that their hormone supply is richer in estrogens.

Even slim women tend to present more marked adipose accumulations on the thighs.

Some factors interfere adversely, causing local alterations that affect the microcirculation of the adipose mass. In time, this leads to anatomical and functional breakdown of the tissue vascular system, which generates problems affecting the hypodermis and the layer immediately above it, namely the dermis.

Cellulite is caused by degeneration of the microcirculation of adipose tissue, with consequent alteration of its most important metabolic functions.

The visible consequence of this tissue degeneration is an increase in the volume of the adipose cells, fluid retention and fluid stagnation in the intercellular spaces.

Cellulite may have genetic (familial predisposition), constitutional, hormonal and vascular causes, often aggravated by a sedentary lifestyle, stress, liver disease, poor diet, intestinal disorders or disorders characterised by marked fluid retention.

Hormonal imbalances (affecting ovarian, pituitary and thyroid hormones) are the cause of cellulite; due to the action of estrogens and their effects on the microcirculation, women are predisposed to this condition, especially during puberty, pregnancy and the pre-menopausal period, when the activity of the ovarian hormones is at a peak. The susceptibility to cellulite is therefore mainly hormone-based rather than genetic.

There is a clear sexual dimorphism in the structural characteristics of subcutaneous connective tissue which predisposes women to develop the irregular extrusions of adipose tissue characteristic of cellulite in the dermis. It has been suggested that the preponderance of antilipolytic activity (α₂-adrenergic-dependent receptor) in female compared with male subcutaneous adipose tissue can promote increased fat deposits in the thighs, and consequently the appearance of cellulite (Rosenbaum et al., Plastic and Reconstructive Surgery, 101(7): 1934-1939, 1998).

Moreover, the lipolytic response to the catecholamines of the adipocytes originating from the subcutaneous adipose tissue of the buttocks, hips or femoral area is lower than that of visceral adipose tissue (Lafontan and Berlan, TIPS, 24: 276-283, 2003), which causes a predisposition to accumulation of adipose tissue in these areas.

At present, cellulite is commonly treated with:

Physical Remedies

Techniques such as electrolipolysis and the more recent laser therapy, ionophoresis, ultrasound therapy and ozone therapy are currently widely used in addition to massage techniques; however, none of these techniques solves the problem at the root.

Diet Supplements

Numerous diet supplements are available on the market (mineral salts, especially potassium, vitamins, ‘fatburner’ or diuretic plant extracts, bowel regulators and bioflavonoids) which claim to increase the metabolism, improve the circulation, protect against cell damage and reduce fat absorption; however, no valid clinical trials are known which support the efficacy of these diet supplements for the treatment of cellulite.

Pharmacologically Active Products

According to a study published in the European J. of Dermatology 10(8) 596-603, 2000, the most common active constituent in the 32 cellulite products analysed is caffeine, present in 14 medicinal products.

Other compounds widely used are:

a) Aminophylline, due to its ability to increase cAMP and lipolysis; both favourable and unfavourable findings about its anticellulite action have been published.

b) Levothyroxine, which exploits the ability of the thyroid hormones to increase the metabolism. In view of the high dose of levothyroxine, systemic absorption may occur, with consequent effects of cardiostimulation and interference with the thyroid, which are particularly harmful in hyperthyroid patients.

c) Escin, due to its vasoprotective heparinoid capacity.

Mesotherapy

This technique involves local intradermal injections of drugs normally administered by the systemic route. It allows a small amount of product to be injected directly into the site of the cellulite; the technique is therefore not systemic and not very invasive. A long-term therapeutic effect can be obtained with mesotherapy because the absorption of the drug at dermal level is slow. The compounds currently used in mesotherapy for the treatment of cellulite are coenzyme A, phosphatidylcholine, aminophylline, escin and homeopathic products.

The adipocyte is a cell which easily modifies its dimensions: with lipogenesis its volume increases, and with lipolysis its volume decreases.

Lipogenesis is produced by LPL (lipoprotein lipase): adipocytes adjacent to the capillaries synthesise and release LPL, which hydrolyses the triglycerides (TG) present in very low density lipoprotein (VLDL) and in the kilomicrons. Glycerol and fatty acids are released, captured by the adipocytes, and esterified into triglycerides.

Lipolysis is produced by hormone-sensitive lipase (HSL): it hydrolyses the TGs present in the adipocytes into free fatty acids and glycerol. The active enzyme is phosphorylated by cAMP-dependent protein kinase A.

cAMP synthesis is dependent on two opposing enzymatic systems
d) adenylate cyclase, which transforms ATP into cAMP. It is negatively regulated by α₂ adrenergic receptors and positively by β adrenergic receptors
e) phosphodiesterase, which breaks down cAMP to AMP inactive on HSL. This activity is inhibited by caffeine, theophylline and aminophylline.

Moreover, the adipocyte secretes numerous factors such as leptin (satiety factor), angiogenic factors (angiotensinogen), prostaglandins PGE₂ (antilipolysis properties) and PGI₂ (cell differentiation), lysophosphatidic acid (cell proliferation) and steroids.

Treatment Strategies

The development of pharmacological bases for cellulite treatment has passed through a number of stages. In the Eighties, inhibition of phosphodiesterase (PDE) with xanthine (caffeine) was used. In the Nineties the problem was tackled by seeking to improve venolymphatic insufficiency with plant extracts having a draining and antioedematous activity (flavonoids and saponosides). More recently, an attempt has been made to restructure the connective tissue with constituents of extracellular matrix and degradation enzymes.

The most promising therapeutic approach, however, seems to be the one designed to increase adipocyte metabolism and lipolysis.

Lipolysis can be induced by: a) stimulation of the β-adrenergic receptors; b) inhibition of the adenosine or α₂-adrenergic receptors; c) inhibition of phosphodiesterase.

A series of clinical trials have evaluated the effects of local application of a β-adrenergic stimulant (isoprenalin), an α₂-adrenergic antagonist (yohimbine) and a phosphodiesterase inhibitor (aminophylline). The results demonstrate that a reduction in localised fat can be obtained pharmacologically, without dieting or exercise (Greenway et al., Obes res, 3: 561S-568S, 1995).

Stimulation of the β-adrenergic receptor increases the cAMP concentration in the adipose cells, thus stimulating lipolysis. Another way of increasing cAMP is to prevent its degradation by inhibiting the enzyme phosphodiesterase.

The most common PDE inhibitors (caffeine, theophylline and aminophylline) are unsatisfactory because of their low specificity and low capacity to be topically absorbed. PDE3, and especially PDE3B, are present in human adipocytes; it consequently appears necessary to inhibit these enzyme sub-types selectively to obtain an effect limited to adipose tissue.

Patents filed on the subject:

EP692250 on the use of flavones to improve the microcirculation GB11588501, FR2797765, EP1261310 and EP1259221 relating to the cosmetic use of xanthine to activate lipase.

From the information given above, it is evident that lipolytic activity is essential, but not sufficient, for the treatment of cellulite. The treatments which have been employed to date, often using phosphodiesterase inhibitors such as xanthine, have not proved wholly satisfactory, so the need for new, effective treatments for this multifactorial morphological alteration of subcutaneous fat is strongly felt.

A group of aryldihydropyridazones and aryldimethylpyrazolones as selective PDE3B inhibitors potentially useful in the treatment of obesity is described in Bioorganic & Medicinal Chemistry Letters, (2003), 13, 3983-3987.

### DESCRIPTION OF THE INVENTION

It has now been found that PDE3 (phosphodiesterase-3) inhibitors, in particular inhibitors of the PDE3B isoform, which is mainly expressed in human adipose tissue, are surprisingly effective against cellulite.

Accordingly, the invention provides the use of a PDE3, preferably a PDE3B inhibitor to prepare a pharmaceutical composition for the topical or mesotherapeutic treatment of cellulite.

Among the PDE3 inhibitors, the compounds anagrelide, cilostazol, pimobendan, milrinone, amrinone, olprinone, enoximone, cilostamide, vesnarinone, trequinsin and their pharmaceutically acceptable salts are preferred. Milrinone, trequinsin and cilostamide are particularly preferred.

A further group of preferred PDE3 inhibitors according to the present invention are the compounds described in Bioorganic & Medicinal Chemistry Letters, (2003), 13, 3983-3987; in particular the compounds:
1) 6-[4-(2-Benzyl-3-oxo-cyclohex-1-enylamino)-phenyl]-5-methyl-4,5-dihydro-2-H-pyridazin-3-one (compound 8a)
2) 3-{2-[4-(4,4-Dimethyl-5-oxo-4,5-dihydro-1-H-pyrazol-3-yl)-2,3-difluoro-phenylamino]-6-oxo-cyclohex-1-enylmethyl}-benzonitrile (compound 1 8n)
3) 5-{4-[2-(2,6-Dichloro-benzyl)-3-oxo-cyclohex-1-enylamino]-2-fluoro-phenyl} -4,4-dimethyl-2,4-dihydro-pyrazol-3-one (compound 18h)
4) 5- [4-(2-benzyl-3-oxo-cyclohex-1-enylamino)-phenyl]-4,4-dimethyl-2,4-dihydro-pyrazol-3-one (compound 18a)
5) 5-{4-[2-(3-nitro-benzyl)-3-oxo-cyclohex-1-enylamino]-2-fluoro-phenyl}-4,4-dimethyl-2,4-dihydro-pyrazol-3-one (compound 18f)
6) 6-[4-(2-Benzyl-3-oxo-cyclohex-1-enylamino)-2-fluoro-phenyl]-5-methyl-4,5-dihydro-2-H-pyridazin-3-one (compound 141)
   As used herein, the term “topical” means designed for or involving local application and action. The topical compositions are preferably in the form of cream, non oily cream, ointment, oil-non oil formulations, gel, spray-gel and patch. For use in mesotherapy the compositions should be in a form suitable for local intradermal injection, preferably in the form of injectable solutions.

The active ingredient is present in concentrations ranging from 0.1 to 3%, preferably from 1 to 2% based on the total composition weight for the topical forms and from 0.1 to 1% by weight for the injectable forms for use in mesotherapy. In addition to PDE3 inhibitors, the compositions according to the invention may contain a compound, a mixture of compounds or an extract active on microcirculation, preferably a saponin or a flavone or extracts containing them. Most preferred are the extracts of Ginkgo biloba, arnica, ananas, dong quai (Angelica siniensis), Centella asiatica, and the saponin escin.

The compound, extract or mixture of substances active on microcirculation are contained in the composition at a concentration of from 0.1 to 4%.

The compositions according to the present invention may further contain pharmaceutically acceptable excipients, such as adjuvants, in particular water or alcohols (ethanol), vitamins, in particular tocopherol, dexpanthenol or retinol palmitate, thickening agents, preservatives, protective colloids, moisturizers, fragrances, electrolytes, moisturizers, gelling agents, agents to increase skin permeability, polymers or copolymers, emulsifiers, emulsion-stabilizing agents and other pharmaceutically acceptable excipients.

Preferred preservatives are substances with low allergenicity such as ethyl alcohol or benzyl alcohol.

The topical formulations can contain oleic acid unsaturated derivatives, such as 10trans-12cis linoleic acid.

Particularly suitable gelling agents are carbomer, more preferably carbomer 940, polyacrylamide isoparaffin-laureth-7, xanthan gum, carrageenin, acacia gum, guar gum, agar gel, alginates and methylhydroxycellulose, carboxymethylcellulose, hydroxypropyl cellulose, hydroxypropyl methylcellulose, hydroxyethylcellulose, ethylcellulose, polyacrylates, polivinilalcool, polyvinylpyrrolidone, colloidal silica. Urea and panthenol are examples of moisturizers according to the invention.

The techniques for the preparation of the pharmaceutical compositions of the present invention are known to those skilled in the art and are described for example in Remington's Pharmaceutical Sciences, XVIII Ed. Mack Publishing Co.

The pharmaceutical formulations containing a PDE3 inhibitor, as described above, are conveniently used for the topical treatment of cellulite in humans, preferably in women.

The following examples illustrate the invention in more detail.

### EXAMPLES

### Example 1

### Non-Oily Cream (% by Weight)

Active ingredient

| | |
|---|---|
| Amrinone | 1 |
| Escin | 2 |

Excipients

| | |
|---|---|
| Glyceryl monostearate | 8 |
| Macrogol cetostearyl ether | 2.5 |
| Liquid petrolatum | 2 |
| White petrolatum | 2 |
| Isopropyl myristate | 4 |
| Myristyl alcohol | 3 |
| p-Hydroxybenzoic acid esters | 0.3 |
| Dep water q.s. to | 100 g |

### Example 1.2

Active ingredient:

| | |
|---|---|
| Milrinone | 1 |

Excipients:

| | |
|---|---|
| Cetostearyl alcohol | 4.5 |
| Glyceryl monostearate | 8.0 |
| Liquid petrolatum | 2 |
| White petrolatum | 2 |
| Dimethicone | 0.30 |
| Isopropyl myristate | 1 |
| Myristyl alcohol | 3 |
| Essential oils | q.s. |
| Dep water q.s. to | 100 g |

### Example 1.3

Active ingredient:

| | |
|---|---|
| Trequinsine | 2 |

Excipients:

| | |
|---|---|
| Oleic acid | 5.0 |
| Macrogol stearate 40 | 9.0 |
| Cetostearyl alcohol | 6.0 |
| Butyl hydroxyanisole | 0.02 |
| Trometamol | 0.1 |
| Dimethicone | 0.3 |
| Carbopol 980 | 0.3 |
| Propylene glycol | 20.0 |
| Sodium sulfite | 0.1 |
| Essential oils | q.s. |
| Dep water q.s. to | 100 g |

### Example 2

### Hydroalcoholic Gel (% by Weight)

Active ingredient:

| | |
|---|---|
| Milrinone | 2 |

Excipients:

| Carbomer | 1.5 | |
|---|---|---|
| Ethyl alcohol 96° EP | 40 | ml |
| Essential oils | q.s. | |
| Triethanolamine | q.s. to adjust pH | |
| Dep water q.s. to | 100 | g |

### Example 3

### Lipophilic Cream (% by Weight)

Active ingredient:

| | |
|---|---|
| Amrinone | 1 |

Excipients:

| | |
|---|---|
| Polygliceryl-3 diisostearate | 4 |
| Glyceryl oleate | 2 |
| Beeswax | 7 |
| Dicapryl ether | 10 |
| Hexyldecanol/hexyldecyl laurate | 10 |
| Glycerin 85% | 5 |
| Magnesium sulfate 7H2O | 1 |
| p-Hydroxybenzoic acid esters | 0.1 |
| Essential oils | q.s. |
| Dep water q.s. to | 100 g |

### Example 4

### Non-Oily Cream (% by Weight)

Active ingredient:

| | |
|---|---|
| 3-{2-[4-(4,4-Dimethyl-5-oxo-4,5-dihydro-1-H-pyrazol- | 1 |
| 3-yl)-2,3-difluoro-phenylamino]-6-oxo-cyclohex- | |
| 1-enylmethyl}-benzonitrile: | |

Excipients:

| | |
|---|---|
| Oleic acid | 5.0 |
| Macrogol stearate 40 | 9.0 |
| Cetostearyl alcohol | 6.0 |
| Butyl hydroxyanisole | 0.02 |
| Trometamol | 0.1 |
| Dimeticone | 0.3 |
| Carbopol 980 | 0.3 |
| Propylene glycol | 20.0 |
| Sodium sulfite | 0.1 |
| Dep. water q.s. to | 100 g |

The following active ingredients can be formulated as non-oily creams according to the Example 4:
6-[4-(2-Benzyl-3-oxo-cyclohex-1-enylamino)-phenyl]-5-methyl-4,5-dihydro-2-H-pyridazin-3-one;
5-{4-[2-(2,6-Dichloro-benzyl)-3-oxo-cyclohex-1-enylamino]-2-fluoro-phenyl}-4,4-dimethyl-2,4-dihydro-pyrazol-3-one;
5-[4-(2-benzyl-3-oxo-cyclohex-1-enylamino)-phenyl]-4,4-dimethyl-2,4-dihydro-pyrazol-3-one;
5- {4-[2-(3-nitro-benzyl)-3-oxo-cyclohex-1-enylamino]-2-fluoro-phenyl}-4,4-dimethyl-2,4-dihydro-pyrazol-3-one;
6-[4-(2-Benzyl-3-oxo-cyclohex-1-enylamino)-2-fluoro-phenyl]-5-methyl-4,5-dihydro-2-H-pyridazin-3-one.

### Example 5

### Injectable Solution for Mesotherapy (5 ml Vial) (the Amounts are Expressed in mg/ml):

Active ingredient:

| | |
|---|---|
| Milrinone | 5 |

Excipients:

| | |
|---|---|
| Sodium metabisulfite, lactic acid | 0.2-0.5 |
| Depurated water q.s. to | 5 ml |

### Example 6

Efficacy Test

Cellulite is a problem with a multifactorial etiology, in which lipogenesis plays a crucial role, In vitro determination of lipolytic activity is therefore an essential factor in the initial screening of potential anticellulite drugs, though totally insufficient for the identification of a final candidate. An in vitro test of lipolytic activity must therefore be associated with a test that evaluates efficacy in vivo.

In Vitro Tests

Determination of extracellular glycerol produced by lipolysis of adipocyte triglycerides.

The in vitro lipolysis test was conducted on human adipocytes obtained from primary cultures of their progenitors, preadipocytes (Promocell), by inducing differentiation. The actual adipocyte differentiation was established by assaying glycerol-3-phosphate dehydrogenase (an enzyme expressed in mature adipocytes, but not in preadipocytes) and the lipids accumulated at intracellular level, with Oil Red 0 (Sigma).

The PDE3 inhibitors were preincubated with the adipocytes at various concentrations for 15 min; 10 nM of isoprenalin, able to induce weak stimulation of lipolysis (25% of the maximum response obtained with isoprenalin 1 μM), was then added. After 4 h the culture medium was removed, and the quantity of intracellular lipids accumulated in each well was determined with Oil Red O.

Lipolytic activity was evaluated by determining the glycerol released into the extracellular medium following triglyceride hydrolysis.

In this test, the formulations with PDE3 inhibitors, including the selective PDE3B inhibitors according to the invention, proved comparable with or more effective than similar formulations containing non-selective PDE inhibitors (caffeine, theophylline).

Effect of Some PDE Inhibitors on Glycerol Release, Stimulated by 10 nM Isoprenalin, in Cultured Human Adipocytes

% INCREASE IN LIPOLYSIS

| | 10 μM |
|---|---|
| Amrinone | 20 ± 6 |
| Anagrelide | 114 ± 11 |
| Cilostazol | 31 ± 9 |
| Enoximone | 65 ± 4 |
| Milrinone | 151 ± 16 |
| Pimobendan | 159 ± 22 |
| Trequinsin | 276 ± 59 |
| Theophylline | 5 ± 4 |
| Caffeine | 10 ± 5 |

Determination of cAMP production in the adipocytes

cAMP is an intracellular messenger, the levels of which depend on its synthesis (adenylate cyclase activity) and breakdown (phosphodiesterase activity). The adipocytes are transferred to a saline solution to which lipolytic agents (adenylate cyclase activators), antilipolytic agents (phosphodiesterase activators) and PDE3 inhibitors are added. After a suitable incubation period, the process is interrupted and the cAMP formed in the cells is extracted. The cell extract is freeze-dried, then taken up and assayed with a specific commercially available colorimetric enzyme immunoassay (EIA) (Amersham, Cayman). The cAMP levels are determined in accordance with the supplier's instructions. In this test, the formulations with PDE3 antagonists according to the present invention proved more effective than similar formulations containing non-selective PDE inhibitors (caffeine, aminophylline).

In Vivo Tests

10 healthy female adults presenting evident cellulite in the upper outer part of the thighs were examined.

Each patient served as her own control for evaluation of efficacy and safety. The cream containing the active constituent (1% Milrinone) was spread on the outer part of one thigh, while the base cream (without the active constituent) was spread in the same area of the other thigh, in a randomised manner.

Treatment with approx. 2-3 cm of cream, massaged in for 2-3 minutes until absorbed, was repeated twice a day every day for 2 months.

During the test, the patients did not follow an exercise programme and were not subjected to any diet restrictions.

The circumference of each thigh was measured periodically, two-thirds of the way between the knee and the greater trochanter, until the end of treatment. The reduction in circumference of the pharmacologically treated thigh compared with the untreated thigh was 2.9-0.7 cm, with a range of 1.3-4.7 cm.

At the end of the observation period, the determination of PDE3 in the blood did not detect the presence of the compound.

1. Pharmaceutical composition for topical administration, containing a PDE3 inhibitor as active ingredient, for use in the treatment of cellulite.

2. Pharmaceutical composition as claimed in claim 1, containing an inhibitor of PDE3B isoform as active ingredient.

3. Composition as claimed in claim 1, wherein said inhibitor is selected from the group consisting of anagrelide, cilostazol, pimobendan, milrinone, amrinone, olprinone, enoximone, cilostamide, vesnarinone and trequinsin.

4. Composition as claimed in claim 3, wherein said inhibitor is milrinone, cilostamide or trequinsin.

5. Composition as claimed in claim 1, wherein said inhibitor is selected from the group consisting of:
a) 6-[4-(2-Benzyl-3-oxo-cyclohex-1-enylamino)-phenyl]-5-methyl-4,5-dihydro-2-H-pyridazin-3-one;
b) 3-{2-[4-(4,4-Dimethyl-5-oxo-4,5-dihydro-1-H-pyrazol-3-yl)-2,3-difluoro-phenylamino]-6-oxo-cyclohex-1-enylmethyl}-benzonitrile;
c) 5-{4-[2-(2,6-Dichloro-benzyl)-3-oxo-cyclohex-1-enylamino]-2-fluoro-phenyl}-4,4-dimethyl-2,4-dihydro-pyrazol-3-one;
d) 5-[4-(2-benzyl-3-oxo-cyclohex-1-enylamino)-phenyl]-4,4-dimethyl-2,4-dihydro-pyrazol-3-one;
e) 5-{4-[2-(3-nitro-benzyl)-3-oxo-cyclohex-1-enylamino]-2-fluoro-phenyl}-4,4-dimethyl-2,4-dihydro-pyrazol-3-one;
f) 6-[4-(2-Benzyl-3-oxo-cyclohex-1-enylamino)-2-fluoro-phenyl]-5-methyl-4,5-dihydro-2-H-pyridazin-3-one.

6. Composition as claimed in claim 1, which is in the form of a gel, spray gel, cream, non-oily cream, oil-non-oil formulation, ointment or sticking plaster.

7. Composition as claimed in claim 1, which in a form suitable for local intradermal injection or mesotherapy.

8. Composition as claimed in claim 7, which is in the form of injectable solution.

9. Composition as claimed in claim 6, wherein the amount of active ingredient is from 0.1 to 3% by weight.

10. Composition as claimed in claim 9, wherein said amount is from 1 to 2% by weight.

11. Composition as claimed in claim 1, wherein said amount is from 0.1 to 1% by weight.

12. Composition as claimed in claim 1, further containing a compound, mixture of substances or extract active on the microcirculation.

13. Composition as claimed in claim 12, containing an extract of arnica, Ginkgo biloba, pineapple, dong quai (Angelica siniensis) or Centella asiatica.

14. Composition as claimed in claim 12, wherein said compound is a saponin or a flavone.

15. Composition as claimed in claim 14, wherein said compound is escin.

16. Composition as claimed in claim 12, wherein the compound, mixture of substances or extract active on the microcirculation is present at a concentration ranging between 0.1 and 4% by weight.

17. A method for treatment of cellulite comprising using a PDE3 inhibitor for the preparation of a pharmaceutical composition and applying the pharmaceutical composition by topical or mesotherapeutic treatment.

18. The method as claimed in claim 17 of a PDE3B inhibitor selected from the group consisting of anagrelide, cilostazol, pimobendan, milrinone, amrinone, olprinone, enoximone, cilostamide, vesnarinone, trequinsin.

19. The method as claimed in claim 17 of a PDE3B inhibitor selected from the group consisting of:
a) 6-[4-(2-Benzyl-3-oxo-cyclohex-1-enylamino)-phenyl]-5-methyl-4,5-dihydro-2-H-pyridazin-3-one;
b) 3-(2-[4-(4,4-Dimethyl-5-oxo-4,5-dihydro-1-H-pyrazol-3-yl)-2,3-difluoro-phenylamino]-6-oxo-cyclohex-1-enylmethyl}-benzonitrile;
c) 5-{4-[2-(2,6-Dichloro-benzyl)-3-oxo-cyclohex-1-enylamino]-2-fluoro-phenyl}-4,4-dimethyl-2,4-dihydro-pyrazol-3-one;
d) 5-[4-(2-benzyl-3-oxo-cyclohex-1-enylamino)-phenyl)-4,4-dimethyl-2,4-dihydro-pyrazol-3-one;
e) 5-{4-[2-(3-nitro-benzyl)-3-oxo-cyclohex-1-enylamino]-2-fluoro-phenyl}-4,4-dimethyl-2,4-dihydro-pyrazol-3-one;
f) 6-[4-(2-Benzyl-3-oxo-cyclohex-1-enylamino)-2-fluoro-phenyl]-5-methyl-4,5-dihydro-2-H-pyridazin-3-one.

20. Composition as claimed in claim 2, wherein said inhibitor is selected from the group consisting of anagrelide, cilostazol, pimobendan, milrinone, amrinone, olprinone, enoximone, cilostamide, vesnarinone and trequinsin.
